(19) United States
(12) Patent Application Publication
(10) Pub. No.: US 2009/0211575 A1
(43) Pub. Date: Aug. 27, 2009

(54) Mask

(75) Inventor: Chih-Cheng Shiue, Taipei (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: QBAS Co., Ltd., Taipei (TW)

(21) Appl. No.: 12/195,836

(22) Filed: Aug. 21, 2008

(30) Foreign Application Priority Data
Feb. 26, 2008 (TW) 97203124
May 23, 2008 (TW) 97209041

(51) Int. Cl. B63C 11/16; A61M 16/00; B63C 11/02; A62B 18/10

(52) U.S. Cl. 128/201.11; 128/204.26; 128/201.27; 128/201.28

(57) ABSTRACT

A mask adapted to be attached to a user's face is disclosed. The mask comprises a body and a gas connection structure. The body is formed with an inner space which is defined along with the user's face. The inner space allows the user to breathe through the nose. The gas connection structure connects to the body so that the inner space is connectable with the ambient air via the gas connection structure. Thereby, the user is capable of breathing through his/her nose to drive the ambient air into the inner space and reduce the moisture formed in the inner space.

This application claims the benefits of the priorities based on Taiwan Utility Mode Applications No. 097203124 filed on Feb. 26, 2008 and No. 097209041 filed on May 23, 2008; the disclosures of the latter are incorporated by reference herein in their entirety.

### CROSS-REFERENCES TO RELATED APPLICATIONS

Not applicable.

### BACKGROUND OF THE UTILITY MODE

1. Field of the Utility Mode

A mask is provided. In particularly, a mask being capable of removing water vapor is provided.

2. Descriptions of the Related Art

Protective masks are commonly applied in underwater or land activities, or in industrial manufacturing to protect a user's face. For example, water goggles may be used in swimming and diving. A schematic view illustrating a user wearing a water goggle A is depicted in FIG. 1. The water goggle A generally comprises a mask body A1, a frame A2 and a lens set A3. The mask body A1 of the water goggle A is adapted to be attached to the user's face to surround the eyes and the nose thereof. In this way, a hermetically sealed inner space (not shown) is formed between the mask body A1 and the user's eyes and nose, so the user's eyes and nose is separated from the water. However, the water goggle A only functions to protect the user's eyes and nose while failing to supply the user with air from the outside. As a result, the user has to breathe through a breathing tube B during an underwater activity. The breathing tube B generally has a tube body B1 and a waterproof device B2. The tube body B1 has two opposite ends, one of which has a mouth-engaging portion B11 while the other one has an air inlet/outlet B12. The waterproof device B2 is disposed at the air inlet/outlet B12. The user bites the mouth-engaging portion B11 into his mouth to inhale or exhale air (and/or liquid) under water to exchange air with the outside via the waterproof device B2 disposed on the other end of the tube body B1 above the water level. Because the water goggle is hermetically attached to the user's eyes and nose, it is unable to exchange any air from the outside. Due to the temperature difference between the inside and the outside of the mask, hot steam exhaled from the nose tends to condense on the interior surface of the lens set of the mask, thus blocking the sight of the user during the underwater activities.

The conventional method of removing the mist or the condensed water (generally referred to as “defogging”) is to take off the water goggle and wash the misted lens by water and replace the water goggle afterwards. Because this method requires the physical action of taking off the water goggle, it is inconvenient for the user. Alternatively, many veteran divers who wear water goggles may rapidly twist the water goggle in the water without taking it off, so that a space is formed between the water goggle and the user's face to allow water to flow therein. After being flushed by water, the water goggle will become clear. Then, with a strong “expelling” action by the nose, the high pressure generated inside the water goggle will force the water and some air out of the water goggle via a one-way valve prearranged below the nose portion or via other sites. Unfortunately, for a rookie diver, this may lead to a risk of water inhalation.

Accordingly, it is highly desirable in the art to make an improvement on the mist problem of the mask in use.

### SUMMARY OF THE UTILITY MODE

One objective of this utility mode is to provide a mask which may effectively reduce, or even eliminate the water vapor inside the mask in use, to prevent misting on an interior surface of lenses thereof which would otherwise block the sight of the user.

To this end, the mask of this utility mode comprises a body and a gas connection structure. The body is adapted to define an inner space along with a user's face to allow the user to breathe through his/her nose. The gas connection structure is connected to the body. The inner space is connected with the ambient air through the gas connection structure so that the user is capable of breathing through the nose thereof to drive the ambient air into the inner space, thereby substantially reducing the water vapor within the inner space.

In one embodiment of this utility mode, the gas connection structure comprises a gas tube with a first end, and a second end opposite the first end. The first end is connected to the body so the inner space is connected with the ambient air through the gas tube.

In another embodiment of this utility mode, the gas connection structure comprises a connecting pipe having a first connecting end and a second connecting end. The first connecting end connects to a breathing tube that is connected with the ambient air. The second connecting end connects to the mask body so that the inner space is connected with the ambient air through the connecting pipe.

The detailed technology and preferred embodiments implemented for the subject utility mode are described in the following paragraphs accompanying the appended drawings for people skilled in this field to well appreciate the features of the claimed utility mode.

### BRIEF DESCRIPTION OF THE DRAWINGS

FIG. 1 is a schematic view of a diving mask and a breathing tube of the prior art;

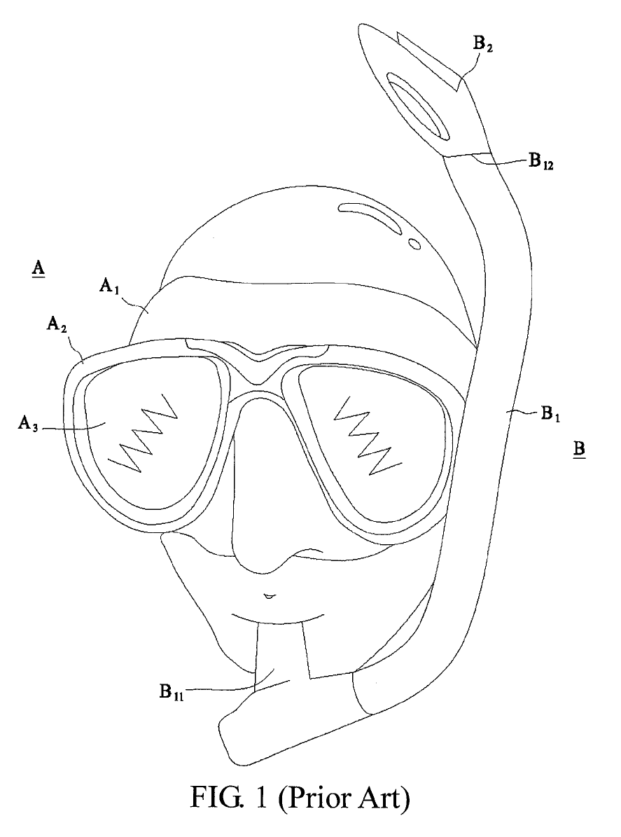

FIG. 2 is a schematic view of a water goggle in a first embodiment of this utility mode;

FIG. 3 is a schematic view of a water goggle in the first embodiment of this utility mode;

FIG. 4 is a cross-sectional view of a water goggle in the first embodiment of this utility mode;

FIG. 5 is an enlarged schematic view of a portion of FIG. 4;

FIG. 6 is a schematic view of a water goggle and a breathing tube in a second embodiment of this utility mode;

FIG. 7 is a schematic view of a connecting pipe and a breathing tube in the second embodiment of this utility mode;

FIG. 8 is a cross-sectional view of a water goggle in the second embodiment of this utility mode; and

FIG. 9 is a schematic view illustrating an air flow path inside the water goggle in the second embodiment of this utility mode.

### DESCRIPTION OF THE PREFERRED EMBODIMENT

The following description is made with reference to a protective mask for swimming or diving, which is commonly known as a water goggle. However, the mask disclosed in this utility mode is not merely limited to use in a water activity such as diving. People skilled in the art, based on the spirit and principle of this utility mode, may appropriately alter or modify the mask into various other protective masks intended to address the fogging problem.

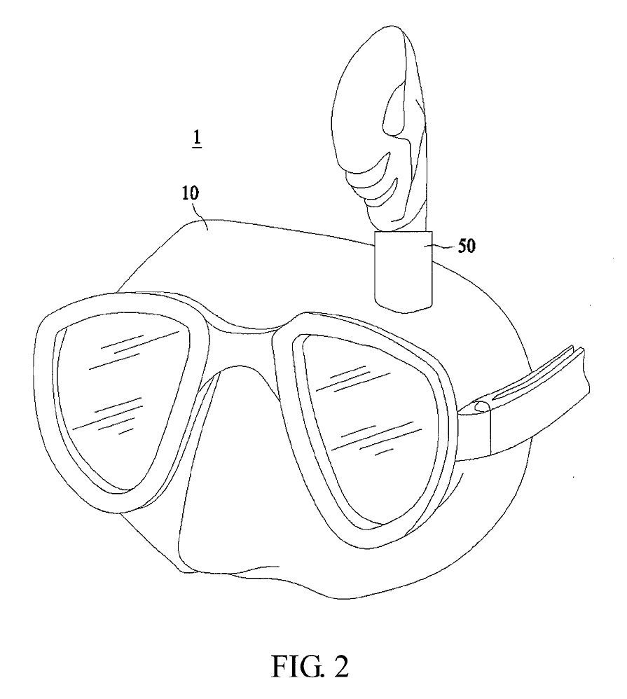

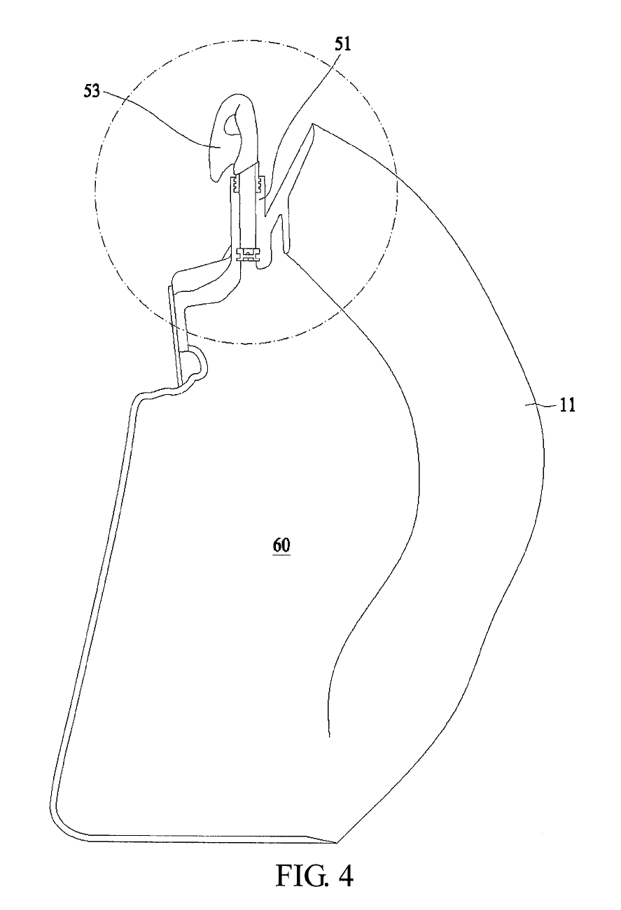

FIG. 2 illustrates a first embodiment of a water goggle according to this utility mode. The water goggle 1 is adapted to be attached to a user's face (not shown), in which the water goggle 1 comprises a body 10 and a gas connection structure 50. The body 10 can be adapted to attached to the user's face to define an inner space 60 (referring to FIG. 4) along with the user's face. The gas connection structure 50 is connected to the body 10 in such a way that the inner space 60 is connected with the ambient air through the gas connection structure 50. Hence, as compared to the prior art, the water goggle of this utility mode allows the user to breathe through his/her nose to drive the ambient air into the inner space, thereby, substantially reducing the water vapor in the inner space.

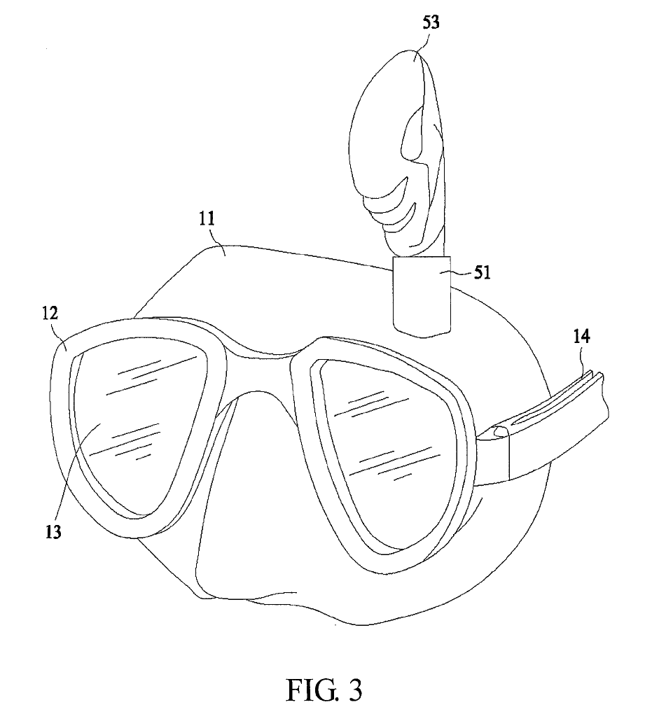

More specifically, the body 10 of the water goggle 1 in this embodiment makes no difference from a water goggle of the prior art. The body 10 generally comprises a mask body 11, a frame 12, a lens set 13 and a band 14, as depicted in FIG. 3. The mask body 10 is attached to the user's face and surrounds a periphery of the frame 12. The frame 12 is adapted to fasten the lens set 13, while the band 14 is adapted to tie the mask body 11 and be hermetically attached to the user's face. The water goggle depicted in the attached drawings of this utility mode is only one example of a wide variety of water goggles, and may be applied to any type or kind of water goggle. For example, the mask body 11 is usually made of a soft material such as silica gel and primarily surrounds the user's eyes and nose. The frame 12 is usually comprised of a main frame and a sub-frame. The lens set 13 is fastened by the frame 12 to provide the user with a clear view underwater. For different variations to and combinations of the frame 12 and the lens set 13, reference may be made to Taiwan Patent Application No. 096213120 owned by the inventor of this application and thus will not be further described herein.

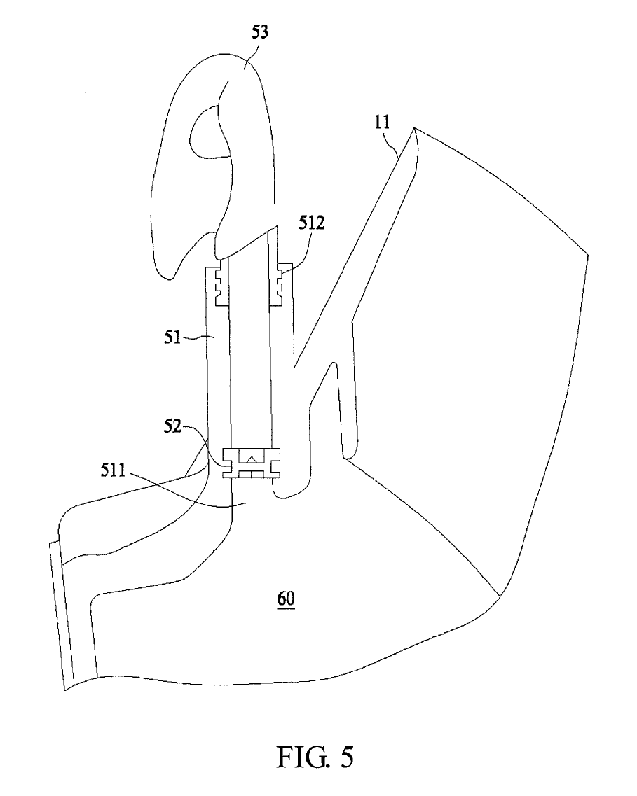

Because the user's body temperature is usually higher than the temperature in an underwater environment, the air exhaled from the user's nose has a temperature higher than that of the surroundings of the mask body 11 when the user engages in an underwater activity wearing the water goggle 1. Consequently, the water vapor present in the inner space 60 tends to condense into water on the lens set 13, which causes misting. However, the water goggle 1 of this utility mode comprises a gas connection structure 50 connected with the body 10, so the dry and cool ambient air can be driven to mitigate the water condensation. More specifically, in reference to FIGS. 2 to 5, the gas connection structure 50 of this embodiment comprises a gas tube 51 with a first end 511 and a second end 512 opposite the first end 511. The first end 511 is connected to the mask body 11 so that the inner space 60 is connected with the ambient air through the gas tube 51. It should be noted that the gas tube 51 does not need to be connected to the mask body 11, and may also be connected to the mask body 11, frame 12 or lens set 13 as long as it enables the inner space 60 to be connectable with the ambient air. Moreover, the material and shape of the gas tube 51 is not subject to any particular limitation, any appropriate material or shape that may accomplish the air connection may be applied in this utility mode. In practical application, the gas connection structure is designed to be detachable, so the user may choose whether to install this structure on the water goggle body depending on the requirements.

In a preferred embodiment of this utility mode, the gas connection structure 50 further comprises a one-way valve 52 and a waterproof device 53. The one-way valve 52 is either disposed on the first end 511 of the gas tube 51 or disposed at the body 10 adjacent to the first end 511, so the ambient air can be driven one way in the inner space 60 formed between the water goggle 1 and the user's face. On the other hand, the waterproof device 53 is disposed on the second end 512 of the gas tube 51. The waterproof device 53 is configured to allow only the ambient air but not any ambient liquid to flow in the inner space 60 through the gas tube 51. This is described in more detail in Taiwan Patent Applications No. 096213122 and No. 096216223 owned by the inventor of this application and thus will not be further described in detail herein. As described above, the user may exchange air with the outside through the gas connection structure 50 when engaging in an underwater activity wearing the water goggle 1 of this embodiment, wherein the outside air is driven by the expelling action of his/her nose. In more detail, when a user inhales air by the nose, moist air originally residing in the inner space 60 of the water goggle 1 is inhaled into the lungs, while the dry and cool ambient air outside the gas connection structure 50 is guided through the waterproof device 53, the one-way valve 52 and the gas tube 51 into the inner space 60 to defog the lens set 13 of the water goggle 1, thereby, allowing the user to maintain a clear view.

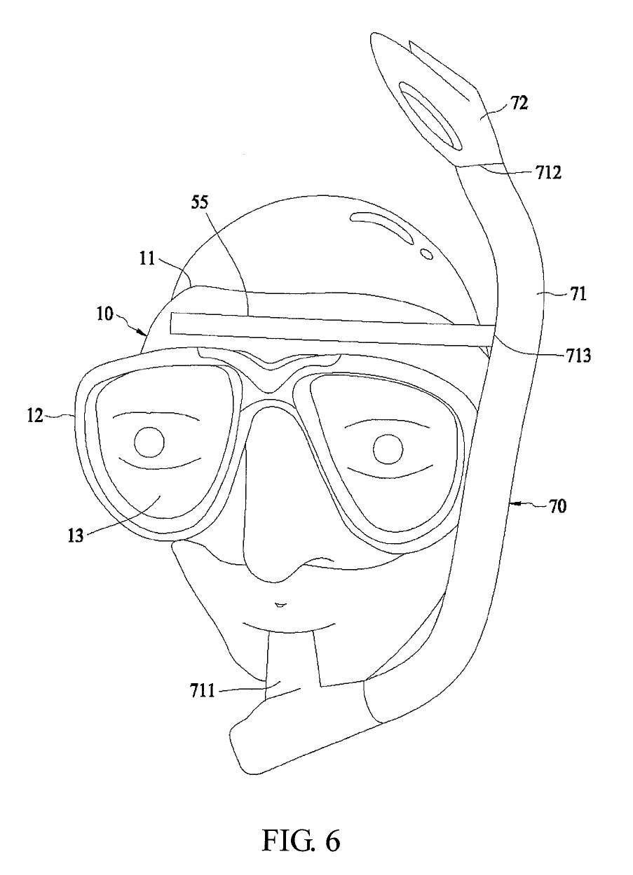

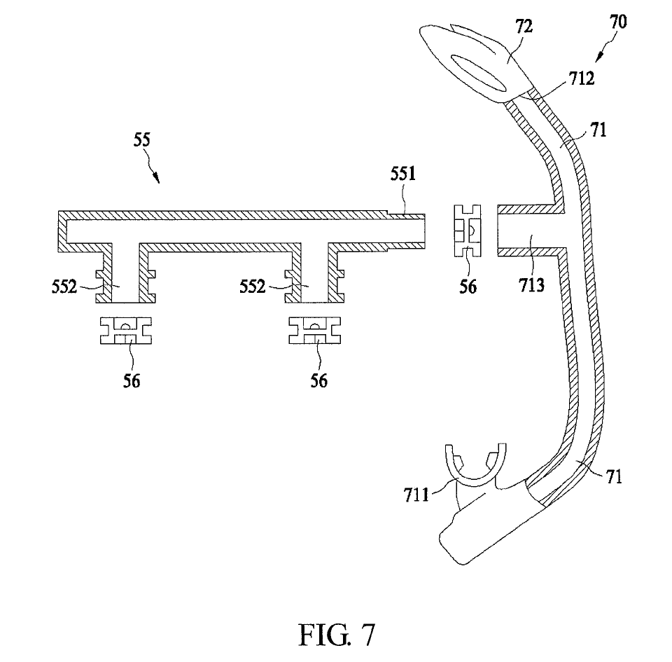

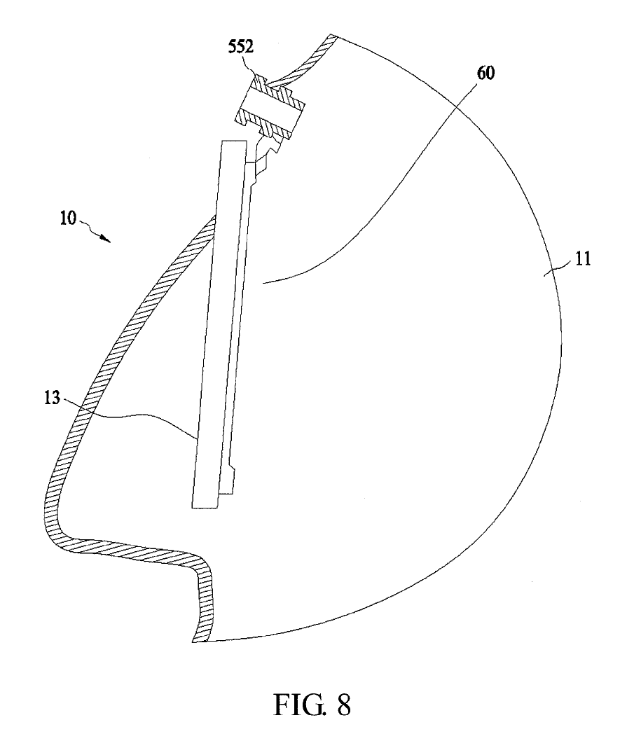

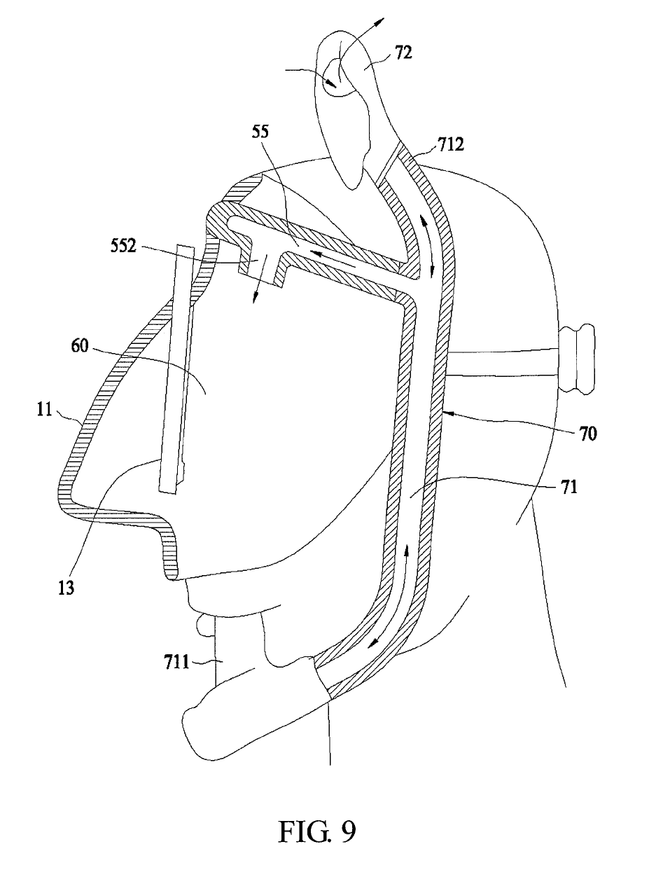

Next, as shown in FIGS. 6 to 9, a second embodiment of the water goggle of this utility mode is depicted therein. Like the previous embodiment, the water goggle 1 of this embodiment also has a body 10 and a gas connection structure 50, in which the body 10 has a mask body 11, a frame 12, a lens set 13 and a band 14. However, unlike the previous embodiment, the gas connection structure 50 of this embodiment must cooperate with a breathing tube 70 for connected to the ambient air. Specifically, the gas connection structure 50 of this embodiment comprises a connecting pipe 55. As shown, the connecting pipe 55 is generally a manifold having a first connecting end 551 and a second connecting end 552. The first connecting end 551 is connected to the breathing tube 70 for connecting to the air outside. The second connecting end 552 is connected to the mask body 11 or connected to either the frame 12 or lens set 13, so that the inner space 60 is connectable with the ambient air through the connecting pipe 55. Similar to the gas tube 51 described above, the material and shape of the connecting pipe 55 is not subject to any particular limitation, and any appropriate material or shape that may accomplish the air connection purpose may be applied in this embodiment. The connecting pipe is designed to be detachable, so that the user may choose whether to install this connecting pipe on the body of the water goggle depending on the requirements.

Furthermore, the breathing tube 70 disclosed in this embodiment is generally the same as those of the prior art; that is, the breathing tube 70 typically has a tube body 71 and a waterproof device 72. The tube body 71 has two opposite ends, one of which is a mouth-engaging portion 711 while the other one is an air inlet/outlet 712. The waterproof device 72 is disposed at the air inlet/outlet 712. When using the water goggle under water, the user bites the mouth-engaging portion 711 into his mouth to inhale or exhale air (and/or liquid) directly through the mouth-engaging portion 711 to exchange air with the outside through the waterproof device 72 disposed at the other end of the tube body 71 above the water level to prevent incursion of ambient liquid into the breathing tube 70. Unlike those from the prior art, the breathing tube 70 of this embodiment is further provided with an opening 713 on the tube body 71 between the mouth-engaging portion 711 and the inlet/outlet 712. The opening 713 is adapted to be hermetically connected to the first connecting end 551 to guide the air flowing from the inlet/outlet 712 into the connecting pipe 55, thereby, substantially reducing the water vapor inside the mask.

In other examples of this embodiment, the gas connection structure 50 further comprises a blocking device 56 disposed at the first connecting end 551 or at the opening 713 of the breathing tube 70 to allow the ambient air to flow in while blocking the incursion of the ambient liquid. The blocking device 56 may be disposed at the second connecting end 552 or at the body 10 in place of the waterproof device to allow the ambient air to flow in while blocking the inflow of the ambient liquid.

As described above, any user wearing the water goggle 1 of this embodiment in an underwater activity can exchange air with the atmosphere primarily through the breathing tube 70. In more detail, when the user inhales air with the mouth through the mouth-engaging portion 711, the ambient air flows through the waterproof device 72 and the tube body 71 into the user's lungs, and when the users exhales air, the air flows out through the waterproof device 72 in the opposite direction. If water vapor condenses on the lens set 13 of the water goggle 1 to block the sight, the user may inhale air through the nose to suck the moist and hot air inside the inner space 60 into the lungs. Meanwhile, the dry and cool ambient air is driven into the inner space 60 through the waterproof device 72, the inlet/outlet 712, the opening 713, the first connecting end 551 and the second connecting end 552. This process may clear the lens set 13 by substantially reducing the humidity level inside the inner space 60. Depending on the practical needs, the user may repeat the inhaling action several times to restore the clear state of the lens set 13 rapidly.

It should be noted that when the user breathes normally through the breathing tube 70, the air is expelled mostly outside via the tube body 71 and the waterproof device 72 directly. In this case, even if a little of the expelled air flows into the connecting pipe 55 and the inner space 60 of the water goggle 1 through the opening 713 to render the water goggle 1 misted, the user may drive the dry and cool ambient air into the inner space 60 simply through the aforesaid inhaling action by the nose. The condensed water on the lens set 13 can still be removed.

In summary, the mask disclosed in this utility mode comprises a gas connection structure adapted to drive the ambient air, so the user may readily inhale the moist and hot vapor inside the mask into the lungs through an inhaling action by the nose and simultaneously drive the dry and cool air into the inner space formed between the mask and the user's face. In this way, the mist on the lens set of the mask caused by temperature difference is eliminated, thus providing the user with a clear sight.

The above disclosure is related to the detailed technical contents and inventive features thereof. People skilled in this field may proceed with a variety of modifications and replacements based on the disclosures and suggestions of the utility mode as described without departing from the characteristics thereof. Nevertheless, although such modifications and replacements are not fully disclosed in the above descriptions, they have substantially been covered in the following claims as appended.

What is claimed is:

1. A mask adapted to attach to a user's face, the mask comprising:
a body, being formed with an inner space which is defined along with the user's face; and
a gas connection structure, being connected to the body, wherein the inner space is connectable with the ambient air through the gas connection structure, thereby the user is capable of breathing by a nose thereof to drive the ambient air into the inner space.

2. The mask of claim 1, wherein the body comprises a mask body, a frame and a lens set, the mask body is attached to the user's face and surrounds a periphery of the frame, and the frame is adapted to fasten the lens set.

3. The mask of claim 2, wherein the gas connection structure comprises a gas tube, having a first end and a second end opposite to the first end, wherein the first end is connected to the mask body so the ambient air is connectable with the inner space.

4. The mask of claim 3, wherein the gas connection structure further comprises a one-way valve disposed at the first end to drive the ambient air into the inner space.

5. The mask of claim 3, wherein the gas connection structure further comprises a waterproof device disposed at the second end to drive the ambient air into the inner space and to prevent ambient liquid form entering.

6. The mask of claim 2, wherein the gas connection structure comprises a connecting pipe, having a first connecting end and a second connecting end, wherein the first connecting end connects to a breathing tube, the breathing tube is connectable with the ambient air, and the second connecting end connects to the mask body so the inner space is connectable with the ambient air through the connecting pipe.

7. The mask of claim 6, wherein the gas connection structure further comprises a blocking device disposed at the first connecting end to allow the ambient air to flow in and to prevent the ambient liquid from entering.

8. The mask of claim 6, wherein the gas connection structure further comprises a blocking device disposed at the second connecting end to allow the ambient air to flow in and to prevent the ambient liquid from entering.
